(19) United States
(12) Patent Application Publication
(10) Pub. No.: US 2005/0239764 A1
(43) Pub. Date: Oct. 27, 2005

(54) Zinc pyrrolidonecarboxylate dihydrate and method of producing the same

(75) Inventors: Eizo Kanatani, Kawasaki-shi (JP); Ryosuke Yumioka, Kawasaki-shi (JP); Shingo Sanada, Kawasaki-shi (JP); Yasushi Kawabata, Kawasaki-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Ajinomoto Co., Inc., Tokyo (JP)

(21) Appl. No.: 11/112,027

(22) Filed: Apr. 22, 2005

(30) Foreign Application Priority Data
Apr. 23, 2004 (JP) 129063/2004

(51) Int. Cl. C07F 3/06; A61K 31/555

(52) U.S. Cl. 514/184; 548/402

(57) ABSTRACT

The present invention aims at providing a convenient method of producing zinc pyrrolidonecarboxylate dihydrate, which is an industrially useful compound, with high optical purity, a higher yield, and better dissolution property. The present invention also provides a convenient method of producing zinc pyrrolidonecarboxylate dihydrate with higher optical purity, a higher yield, and better solution property by adding a zinc salt to an aqueous medium containing a salt of pyrrolidonecarboxylic acid as a starting material and separating a crystal from the aqueous medium at a specific pH.

### FIELD OF THE INVENTION

The present invention relates to a zinc pyrrolidonecarboxylate dihydrate with drastically improved solution properties and higher optical purity, a method of producing the same (one pot production method), and an evaluation method thereof.

### BACKGROUND OF THE INVENTION

Pyrrolidonecarboxylic acid is a main component of a water-soluble substance, called as a Natural Moisturizing Factor (NMF), in stratum corneum. It is well known that the pyrrolidonecarboxylic acid usually remains salt in stratum corneum and thus plays an important role in moisture retention. Various salts and derivatives of the pyrrolidonecarboxylic acid have been examined from old times. Such salts and derivatives have been widely used in the field of pharmaceutical agents, the field of food, the industry, the cosmetic•toiletry field, or used as a production intermediate of materials used in the above-described fields.

Among various salts, zinc pyrrolidonecarboxylate (zinc pidolate) is very useful compound industrially presenting broader functions such as the astringent effect, bacteriostatic action, an odor prophylaxis component, a production intermediate for an optical active glutamic acid, and a stabilizing effect of polyvinyl chloride resin. Up to the present, only a few examples for the method of producing the zinc pyrrolidonecarboxylate as products or a production intermediate have been disclosed.

For example, in ES8604138, zinc L-pyrrolidonecarboxylate dihydrate is obtained as following procedures: adding aqueous zinc acetate solution into L-pyrrolidonecarboxylic acid (free acid) aqueous solution and then adding an acetone thereto during recrystallization. However, using L-pyrrolidonecarboxylic acid (free acid) as starting material, zinc acetate which is hard to be obtained industrially as a zinc source is used. Also, an acetone, an organic solvent, must also be added as a poor solvent and then cooled to −20° C. during recrystallization. Therefore, this method is not always satisfactory as an industrially convenient production method.

In addition, JP-A-3-168240 discloses an example that zinc DL-pyrrolidonecarboxylate dihydrate is manufactured as followings: reacting a DL-pyrrolidonecarboxylic acid (free acid) with a zinc oxide in water and then conducting an agitating cooling crystallization. In this method, a zinc oxide which hardly dissolves in water under a neutral region is used. Higher temperature condition of 90° C. or more is also required, and a yield is not more than 48%. Therefore, this method is also not always satisfactory as an industrially convenient production method.

JP-B-43-27859 also discloses an optical separating method using a zinc salt of pyrrolidonecarboxylic acid, which is a useful seasoning or intermediate of optical active glutamic acid. Specifically, aqueous solution of zinc sulfate is mixed with aqueous solution of sodium hydroxide of a DL-pyrrolidonecarboxylic acid. And then, a crystal of zinc L-pyrrolidonecarboxylate dihydrate is added thereto as a crystal seed, and then crystal is collected using a cooling crystallization from 55° C. With such method, zinc pyrrolidonecarboxylate dihydrate having an optical purity of 93.8% is obtained. Even though the crystal seed and the cooling crystallization are required, as it's yield is extremely low to about three times an added crystal seed and products also generate an unexpected precipitation in the aqueous solution, the product quality (solution property) is not always satisfactory.

As described above, it has not yet been disclosed the convenient method of producing zinc pyrrolidonecarboxylate dihydrate, as an industrially useful compound with higher optical purity, higher yield, and better solution properties.

### SUMMARY OF THE INVENTION

An object is therefore to provide a convenient method of producing zinc pyrrolidonecarboxylate dihydrate, with higher optical purity, a higher yield, and better solution properties.

Inventors of the present invention have made a hard working to achieve the aforementioned object. As a result, a method of producing zinc pyrrolidonecarboxylate dihydrate with higher optical purity, higher yield, and better solution property is found by adding a zinc salt in aqueous medium containing a salt of pyrrolidonecarboxylic acid as starting materials and separating a crystal from the aqueous medium having a specific pH, and thus the present study comes to be completed.

That is, the present invention includes the following aspects.

(1) A method of producing zinc pyrrolidonecarboxylate dihydrate, which comprises the steps of
adding a zinc salt to a solution of a salt of pyrrolidonecarboxylic acid in an aqueous medium to allow reaction of the zinc salt with the salt of pyrrolidonecarboxylic acid to give precipitated crystals of zinc pyrrolidonecarboxylate dihydrate in the aqueous medium, and separating the crystals of zinc pyrrolidonecarboxylate dihydrate from the aqueous medium at a pH ranging from 2.8 to 4.2 (conversion value at 25° C.).

(2) The production method described in (1), wherein the salt of pyrrolidonecarboxylic acid has an optical purity ranging from 100% to 50%.

(3) The production method described in (2), further comprising a step of obtaining the salt of pyrrolidonecarboxylic acid with an optical purity ranging from 100 to 50%, by heating a salt of glutamic acid with an optical purity ranging from 100 to 50%, in the aqueous medium.

(4) The production method described in any one of (1) to (3), wherein the zinc salt is at least one selected from the group consisting of a zinc chloride and a zinc sulfate.

(5) A zinc pyrrolidonecarboxylate dihydrate with an optical purity ranging from 100 to 95%, which is obtained by the production method described in any of (1) to (4).

(6) A method of evaluating a zinc pyrrolidonecarboxylate dihydrate, which comprises a step of measuring light transmittance of a 10 g/dl aqueous solution thereof.

(7) The zinc pyrrolidonecarboxylate dihydrate described in (5), wherein the light transmittance of a 10 g/dl aqueous solution thereof at a wavelength of 550 nm is within the range of 94 to 100% under an optical path length of 10 mm.

(8) A zinc pyrrolidonecarboxylate dihydrate with an optical purity ranging from 100% to 95%, wherein a light transmittance of a 10 g/dl aqueous solution thereof at a wavelength of 550 nm is within the range of 94 to 100% under an optical path length of 10 mm.

(9) A cosmetic comprising the zinc pyrrolidonecarboxylate dihydrate described in (5).

With the present invention, a simple method of producing zinc pyrrolidonecarboxylate dihydrate, an industrially useful compound, with a higher optical purity, a higher yield, and a better solution properties can be provided.

### BRIEF DESCRIPTION OF THE DRAWINGS

FIG. 1 is an observational view of product solution properties (from the left, Example 2, Example 1, Comparative Example 2, Comparative Example 1).

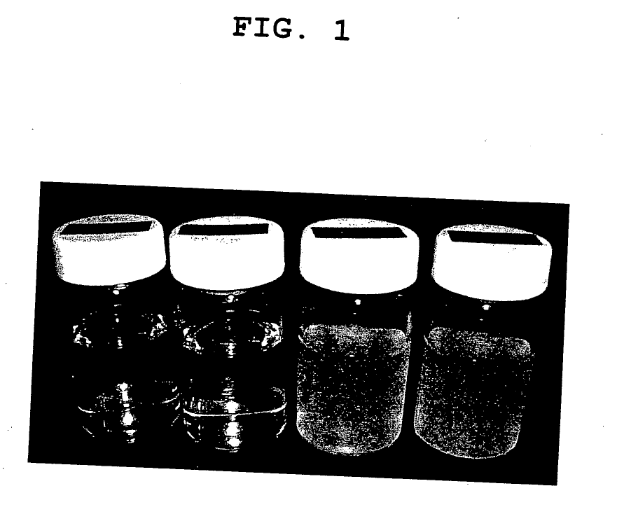

### DETAILED DESCRIPTION OF THE INVENTION

Hereinafter, the present invention will be explained in detail.

According to the present invention, a method of producing zinc pyrrolidonecarboxylate dihydrate essentially comprises [Second Step] described below, and preferably, both [First Step] and [Second Step].

The [First Step] is a step of obtaining salt of pyrrolidonecarboxylic acid by heating salt of glutamic acid as starting material and base in aqueous medium.

The [Second Step] is a step of obtaining zinc pyrrolidonecarboxylate dihydrate by separating crystal, which is obtained by adding a zinc salt into aqueous medium containing a salt of pyrrolidonecarboxylic acid as a starting material, from the aqueous medium in pH ranging from 2.8 to 4.2 (conversion value at 25° C.).

Each step will be described below in order.

As disclosed in JP-A-2003-34680, the first step the present invention optionally has may be a conventional method including a step of reacting salt of glutamic acid in a condition of higher temperature and higher pressure. To achieve an increased yield and improved optical purity of a salt of pyrrolidonecarboxylic acid, it is desirable to choose a method having lower thermal history.

An embodiment of the reaction to be used in the first step is, considering the lower thermal history, preferably an embodiment of conducting cyclization using a flow method under treatment of pressure and temperature. In a batch method using autoclave and the like, it is desirable to choose an embodiment of lower thermal history, such as thermal reaction in basic aqueous medium of a salt of glutamic acid. There is no particular limitation with material and the shape of a reactor, and it is proper to choose a material, such as stainless, resistive to pressure and temperature of the reactor, and the reactor may have an inner volume, a thickness of a wall and opening radius suitable for absorbing pressure variation.

Optical purity of glutamic acid of salt of glutamic acid which is a starting material in the first step is generally in the range of 100 to 50%. To afford zinc pyrrolidonecarboxylate having a higher optical purity, the optical purity of the glutamic acid is preferably in the range of 100 to 60%, more preferably 100 to 70%, still more preferably 100 to 80%, and particularly preferably 100 to 90%. The optical purity herein represents an abundance difference between L-form and D-form. For example, an optical purity of 70% represents that L-form of 85% and D-form of 15% are contained.

There is no particular limitation with a method of producing glutamic acid of salt of glutamic acid which is starting materials in the first step. For example, in case of a microorganism fermentation method, treacle using sugar cane, corn, cassava as starting materials is generally used as glycogen, and then inorganic nitrogen source is added thereto so as to make a medium, and then microorganism is fermented in the medium to accumulate glutamic acids, and then the accumulated glutamic acids are neutralized, separated, purified, and crystallized, thereby obtaining glutamic acid.

Regarding a type of salts of the salt of glutamic acid which is starting materials in the first step, there is no particular limitation except zinc salt, and specifically, alkali metal salts such as sodium salts, potassium salts and the like, alkali earth metal salts such as calcium salts, magnesium salts and the like, organic base salts such as ethanol amine salts, diethanol amine salts and the like, basic amino acid salts such as lysine salts, arginine salts and the like can be used. These may be used alone or in a mixture of two or more kinds thereof.

With regard to the aqueous medium used in the first step, main solvent is water and it is possible to contain cosolvent. As long as the cosolvent is not reacted with substrates under the reaction condition, there is no particular limitation, and specifically, alcohol such as methanol, ethanol, t-butanol, propylene glycol and the like, ketones such as acetone, methylethylketone and the like, and the like can be used. These may be used alone or in a mixture of two or more kinds thereof. To simplify the reaction system, it is desirable to reduce the cosolvent if possible, and use of water alone is most preferable. As long as the reaction is not adversely affected, water is not limited, and specifically, city water, industrial water, ion exchange water, distilled water and the like are used. To simplify the reaction system, it is desirable to use water containing less impurity, more preferably, ion exchange water or distilled water.

As long as the cyclization reaction to give a salt of pyrrolidonecarboxylic acid proceeds, the base in the first step is not particularly limited, and specifically, metal hydroxide such as sodium hydroxide, potassium hydroxide and the like, organic base such as ethanolamine, diethanolamine, triethanolamine and the like, and basic amino acid such as lysine, arginine, ornithine and the like can be used. These may be used alone or in a mixture of two or more kinds thereof. For easy progress of a salt exchange reaction with a zinc salt in a subsequent step, sodium hydroxide and potassium hydroxide are preferably used.

As long as a cyclization reaction proceeds, the pH in the first step is not limited and is generally in the range of 6.0 to 12.0, and, to prevent or reduce racemization, it is referably 6.0 to 9.0, more preferably 6.0 to 8.0, and still ore preferably 6.0 to 7.0.

As long as a cyclization reaction proceeds, the reaction temperature in the first step is not particularly limited, and it is generally in the range of 80 to 300° C. When the temperature is lower than 80° C., the reaction obviously becomes markedly slow, and on the contrary, when the temperature is higher than 300° C., racemization proceeds. For stable progress of the reaction without racemization, the temperature is preferably in the range of 100 to 270° C., more preferably 150 to 250° C., and still more preferably 170 to 220° C.

As for the pressure in the first step, it is subject to no particular limitation as long as it is higher than the vapor pressure at the reaction temperature to be applied and the reaction proceeds. It is generally in the range of 0.1 MPa to 25 MPa. To prevent racemization, the pressure is preferably 0.5 to 20 MPa, more preferably 0.5 to 15 MPa, and still more preferably 0.5 to 10 MPa.

As long as the cyclization reaction proceeds, the reaction concentration in the first step is not particularly limited, and it is generally in the range of 25 to 75 wt % as pyrrolidonecarboxylic acid (free acid). When the concentration is lower than 25 wt %, the production efficiency or yield is lowered, and on the contrary, when it is higher than 75 wt %, the viscosity of the reaction system remarkably increases to degrade the operability. For stable and high yield production of the salt of pyrrolidonecarboxylic acid, the concentration is preferably in the range of 30 to 70 wt %, more preferably 40 to 60 wt %, and particularly preferably 45 to 55 wt %.

The reaction time in the first step is not particularly limited as long as the reaction completes. It is generally in the range of 10 sec to 24 hr. While subject to change depending on the reaction temperature to be employed, a longer reaction time is associated with progress of racemization as a side reaction. Therefore, it is desirable to set the reaction time in consideration of the temperature history of the whole reaction including cooling to room temperature and the like.

To prevent the first step from becoming a rate-limiting step in an industrial production, the reaction time is preferably 30 min to 12 hr, more preferably 1 to 8 hr, and particularly preferably 2 to 6 hr.

After the reaction of the first step, the obtained reaction solution can be used in the next step. By going through such first step, it is possible to obtain a salt of pyrrolidonecarboxylic acid having an optical purity of about 100 to 50%.

The second step, which is inevitably comprised in the method of the present invention, will be described.

The second step comprises a step of adding zinc salt into solution dissolving a salt of pyrrolidonecarboxylic acid in the aqueous medium so as to react the salt of pyrrolidonecarboxylic acid with the zinc salt and thus precipitating a crystal of zinc pyrrolidonecarboxylate hydrate in the aqueous medium, and a step of separating the crystal of the zinc pyrrolidonecarboxylate dihydrate from the aqueous medium at a pH ranging from 2.8 to 4.2 (conversion value at 25° C.)

Regarding the salt of pyrrolidonecarboxylic acid which is used as starting materials in the second step, although the history is particularly not restricted, it is out of the question to use any one obtained by the first step, a different method, or purchasing. Optical purity of the salt of pyrrolidonecarboxylic acid is generally in the range of 100 to 50%. To efficiently yield zinc pyrrolidonecarboxylate having higher optical purity, the optical purity is preferably in the range of 100 to 60%, more preferably 100 to 70%, still more preferably 100 to 80%, and particularly preferably 100 to 90%.

Regarding a type of salts of the salt of pyrrolidonecarboxylic acid which is a starting material in the second step of the present invention, there is no particular limitation except zinc salt, and specifically, alkali metal salts such as sodium salts, potassium salts and the like, alkali earth metal salts such as calcium salts, magnesium salts and the like, organic base salts such as ethanol amine salts, diethanol amine salts and the like, basic amino acid salts such as lysine salts, arginine salts and the like, and the like can be used. These may be used alone or in a mixture lo of two or more kinds thereof. When the second step is carried out after the first step, the salt of pyrrolidonecarboxylic acid obtained in the first step is preferably used as it is.

With regard to the aqueous medium used in the second step of the present invention, main solvent is water and it is possible to contain cosolvent. As long as the cosolvent is not reacted with substrates under the reaction condition, the cosolvent is not particularly limited, and specifically, alcohols such as methanol, ethanol, t-butanol, propylene glycol and the like, ketones such as acetone, methylethylketone and the like can be used. These may be used alone or in a mixture of two or more kinds thereof. To simplify the reaction system, it is desirable to reduce the cosolvent if possible, and use of water alone is most preferable. As long as the reaction is not adversely affected, water is not limited, and specifically, city water, industrial water, ion exchange water, distilled water and the like are used. To simplify the reaction system, it is desirable to use water containing less impurity, more preferably, ion exchange water or distilled water.

Regarding the zinc salt to be used in the second step of the present invention, as long as the zinc salt dissolves in water under the pH region of reaction, the zinc salt not particularly limited, and inorganic zinc such as zinc sulfate, zinc chloride and the like, and organic zinc such as zinc gluconate, zinc paratoluenesulfonate and the like are specifically used. These may be used alone or in a mixture of two or more kinds thereof. Since salts except the zinc pyrrolidonecarboxylate dissolve well and are hardly mixed with a crystal of zinc pyrrolidonecarboxylate dihydrate, it is desirable to use an inorganic salt, and more desirably, at least one selected from the group consisting of zinc chloride and zinc sulfate. Furthermore, to achieve a rapid salt-exchange reaction with a salt of pyrrolidonecarboxylic acid, it is particularly desirable to use zinc sulfate. The mode of addition of zinc salt to a solution in which a salt of pyrrolidonecarboxylic acid has been dissolved, is not particularly limited as long as the salt-exchange reaction proceeds, and the zinc salt may be added in the state of a solid or may be previously dissolved in the aqueous medium described above. To achieve a rapid salt-exchange reaction, it is desirable to dissolved the zinc salt in advance in the aqueous medium described above before addition.

While the concentration of a solution, in which a salt of pyrrolidonecarboxylic acid has been dissolved, before addition of the zinc salt in the second step of the present invention, is not particularly limited as long as the reaction proceeds, it is generally 20 to 75 wt % based on pyrrolidonecarboxylic acid (free acid). When it is lower than 20 wt %, production efficiency and yield are degraded, and on the contrary, when it is higher than 75 wt %, viscosity of the reaction system remarkably increases to degrade operability, thus increasing the possibility of affecting the optical purity. To obtain zinc pyrrolidonecarboxylate stably in a higher yield, the concentration is preferably in the range of 25 to 70 wt %, more preferably 35 to 60 wt %, and particularly preferably 45 to 55 wt %.

In the second step of the present invention, zinc salt is used in an amount corresponding to 0.1 to 0.9 mol of zinc ion per 1.0 mol of pyrrolidonecarboxylic acid (free acid). When the amount of zinc ion is less than 0.1 mol relative to 1.0 mol of pyrrolidonecarboxylic acid (free acid), the yield is likely to decrease, and on the contrary, when it is more than 0.9 mol, the content of impurity in the product increases to degrade the product. To achieve high yield and high quality of dissolution state, the amount of zinc ion is preferably 0.2 to 0.8 mol, more preferably 0.3 to 0.7 mol, and particularly preferably 0.4 to 0.6 mol, relative to 1.0 mol of pyrrolidonecarboxylic acid (free acid).

According to the present invention, pyrrolidonecarboxylates are generally contained in a proportion of 10 to 70 wt % upon conversion to a free acid, during crystal separation. When the content is lower than 10 wt %, production efficiency and the yield of the crystal becomes low, and when the content is conversely higher than 70 wt %, the viscosity of the reaction system strikingly increases to degrade operability, which in turn highly possibly affects the optical purity. To achieve stable production of zinc pyrrolidonecarboxylate in a high yield at a high optical purity, 15 to 60 wt % is preferable, 20 to 50 wt % is more preferable and 25 to 40 wt % is particularly preferable.

While the reaction temperature of the zinc salt exchange reaction in the second step in the present invention is not particularly limited as long as the exchange reaction proceeds, it is generally −10° C. to 100° C. A temperature lower than −10° C. markedly delays freezing of the system and progress of the reaction, and a temperature higher than 100° C. may allow progress of racemization or decomposition. To achieve stable progress of salt exchange, 0° C. to 70° C. is preferable, 5° C. to 50° C. is more preferable and 10° C. to 30° C. is particularly preferable.

In the second step of the present invention, pH before zinc salt addition indicates pH before adding the zinc salt in the salt of pyrrolidonecarboxylic acid solution, and typically is adjusted to be in the range of pH 4.0 to 5.5 so as to improve quality of products and yield, preferably pH 4.0 to 5.5, more preferably pH 4.5 to 5.3, and particularly preferably pH 5.0 to 5.2. The pH before zinc salt addition can be properly controlled according to base type or quantity in the first step and acid addition described below.

In the second step of the present invention, pH after zinc salt addition indicates pH at the time precipitation of zinc pyrrolidonecarboxylate dihydrate crystal has been completed at a zinc salt exchange reaction temperature. In practice, it indicates a pH when it substantially ceased to vary in continuous measurement of pH starting from the addition of zinc salt. While the pH may vary depending on the temperature, the pH after zinc salt addition is generally adjusted to fall within the range of pH 2.8 to 4.3, preferably pH 3.3 to 4.2, more preferably pH 3.5 to 4.1, and particularly preferably pH 3.8 to 4.0, so as to improve quality and yield of the product. The pH after zinc salt addition can be properly controlled by the kind and amount of the base in the first step and addition of acid to be described below.

In the present invention, it is important to separate zinc pyrrolidonecarboxylate dihydrate crystal from aqueous medium at pH (conversion value at 25° C.) ranging 2.8 to 4.2 so as to improve quality and yield of the product. When pH is less than 2.8, crystallization rate is obviously deteriorated while solution properties of products is improved, on the contrary, when pH is higher than 4.2, solution property of products is remarkably deteriorated while higher yield is secured. To secure the quality of products and increase the yield, the pH is preferably in the range of 3.3 to 4.2, more preferably 3.5 to 4.2, still more preferably 3.5 to 4.1, further more preferably 3.5 to 4.0, and particularly preferably 3.8 to 4.0. While the pH may vary depending on the measurement temperature or ion type, so as to control the pH to be in the range described above, it is possible to properly control the pH based on the detailed data or chemical equilibrium theory.

In the second step of the present invention, it is desirable to minimize the difference between the pH after zinc salt addition described above and the pH when the zinc pyrrolidonecarboxylate dihydrate crystal is separated from the aqueous medium, and the difference is preferably in the range of −0.5 to +0.5, more preferably −0.3 to +0.3, and more preferably −0.1 to +0.1. To make the difference substantially zero in the second step, it is only necessary to separate crystal from the aqueous medium without changing the pH after precipitation of the zinc pyrrolidonecarboxylate dihydrate crystal in the aqueous medium. That is, after precipitating the zinc pyrrolidonecarboxylate dihydrate crystal in the aqueous medium, addition of acid or alkali is desirably avoided.

In the second step of the present invention, the acid is subject to no particular limitation as long as it is capable of adjusting the pH to fall within the range for the reaction system, and it is specifically possible to use inorganic acids such as hydrochloric acid, sulfuric acid, nitric acid, phosphoric acid and the like, and organic acids such as acetic acid and the like. These may be used alone or in a mixture of two or more kinds thereof. To achieve a desired pH by means of addition of only a small amount and reduction of the ion species in the reaction system, it is desirable to use an inorganic acid, more preferably hydrochloric acid, sulfuric acid and nitric acid, and more preferably nitric acid. In order to prevent the zinc pyrrolidonecarboxylate dihydrate from being mixed with impurity, it is desirable to control pH by adding required acid before zinc salt addition or after zinc salt addition, most desirable to control pH by adding an acid before zinc salt addition.

The reaction time of salt exchange by zinc salt in the second step of the present invention varies depending on the acid, pH and temperature to be used. The reaction time is not subject to any particular limitation as long as the reaction can be completed, and it is generally in the range of 5 min to 3 hr at ambient temperature. To afford sufficient aging effect to produce easily separable crystal, the reaction time is preferably in the range of 10 min to 2 hr, and more preferably 20 min to 1 hr. The reaction time here indicates the time period between addition of zinc salt and when the pH substantially ceases to change at the reaction temperature.

In the second step of the present invention, it is possible to use an ordinary method to allow precipitation of crystals, and specifically cooling crystallization, agitation crystallization method and the like are used. For crystal separation, conventional methods such as centrifugal separation, filtration and the like are used.

In the second step of the present invention, the temperature when crystal is separated from the aqueous medium is preferably made lower so as to decrease solubility of crystal thereby increasing yield, and conventionally in the range of −10 to 50° C. When lower than −10° C., the whole reaction system is frozen or viscosity is increased such that it is hard to carry out separating operation, on the contrary, when higher than 50° C., the solubility of the zinc pyrrolidonecarboxylate dihydrate is increased such that the yield is deteriorated. To achieve better operability and higher yield, the temperature is preferably in the range of −5 to 40° C., more preferably 0 to 30° C., still more preferably 5 to 25° C., and particularly preferably 5 to 20° C.

The above-described aqueous medium can be used as aqueous medium for washing the crystals separated in the second step of the present invention. It is desirable to use larger amount of aqueous medium for washing in order to obtain a product with higher quality. Generally, therefore, dry crystal of zinc pyrrolidonecarboxylate dihydrate is washed with 5 to 150 wt % of aqueous medium. Since zinc pyrrolidonecarboxylate dihydrate is highly water-soluble, when it is washed with aqueous medium in an amount larger than 150 wt %, the separated crystal is easily dissolved to lower the yield, and when it is washed with aqueous medium in an amount less than 5 wt %, impurities such as ion and the like remain in the product, which prevents preparation of a product with higher quality. To achieve higher yield and higher quality, the amount of aqueous medium to be used for washing is preferably 5 to 100 wt %, more preferably 10 to 90 wt %, and particularly preferably 20 to 80 wt %, relative to the crystal. The aqueous medium for washing preferably has a temperature range selected from the above-described separation temperature. To prevent re-adhesion of impurities that degrade the dissolution state, moreover, washing is preferably performed at a pH for crystal separation.

The crystal obtained by separating in the second step is dried by conventional methods to remove water adhered to the crystal. The drying temperature can be set in consideration of the decompression degree, ventilation ability and heating ability of a drying device, and generally set to 30 to 140° C. When the temperature is lower than 30° C., drying is difficult. Thus, it is necessary to increase ventilation or decompression. On the contrary, when the temperature is higher than 140° C., the crystal loses hydration water and thus becomes an anhydrate having high hygroscopicity. To achieve industrially stable production, the temperature is preferably in the range of 50 to 120° C., more preferably 70 to 100° C., and still more preferably 70 to 90° C.

According to the second step of the present invention, if proper condition is selected, it is possible to obtain zinc pyrrolidonecarboxylate dihydrate with high optical purity of generally not less than 95%, in a high yield of, for example, not less than 40%. The zinc pyrrolidonecarboxylate dihydrate obtained by the method of the present invention has high quality, and, for example, a solution obtained by dissolving the dihydrate in water has high light transmittance. Using the obtained zinc pyrrolidonecarboxylate dihydrate as an evaluation subject, when a solution (40 ml) obtained by dissolving 4 g of the evaluation subject in water, that is, an aqueous solution containing 10 g/dl of the evaluation subject, is measured in 10 mm optical length using a light with 550 nm wavelength, transmittance is preferably in the range of 94 to 100%. Specifically, when the pH during crystal separation is set to the desirable range, for example, a crystal with optical purity of not less than 98% and transmittance of not less than 98% is obtained in a yield of not less than 50%.

The inventors of the present invention have found that light transmittance in the solution of the evaluation subject is measured as a quality evaluation method for zinc pyrrolidonecarboxylate dihydrate, as described above. In such evaluation method, the transmittance of solution, which is obtained by dissolving the zinc pyrrolidonecarboxylate dihydrate in water at desired concentration, is measured. The concentration of the evaluation subject is illustrated to be in the range of 5.0 to 15.0 wt %, and specifically is 10 g/dl (e.g.: evaluation subject 4 g is dissolved in water to be 40 ml) as described above. When measuring transmittance, the light is generally visual light, and specifically the wavelength thereof is 550 nm. Examples for specific evaluation will be described in detail the following Examples.

When the first and second steps of the present invention are continuously carried out, the simplest method to obtain zinc pyrrolidonecarboxylate dihydrate with a high optical purity in a high yield comprises adding, after pH adjustment, an aqueous solution of zinc salt directly at room temperature to a reaction solution obtained by reacting a salt of glutamic lo acid in an autoclave to give a salt of pyrrolidonecarboxylic acid, and after completion of the reaction, separating, washing and drying the crystal.

The zinc pyrrolidonecarboxylate dihydrate obtained from the present invention can be used as cosmetic or medicine such as astringent, antibacterial, odor preventing agent and the like. Further, it is possible to use as a production intermediate of optical active glutamic acid. It is also possible to use as stabilizing agent for polyvinyl chloride resin.

The cosmetic is composition which is constructed to be applied to human skin or hair. The cosmetic of the present invention comprises inevitably the zinc pyrrolidonecarboxylate dihydrate obtained from the present invention, and may contain aqueous, oil, powder components and the like. According to the present invention, the zinc pyrrolidonecarboxylate dihydrate content of the whole cosmetic is preferably in the range of 0.05 to 5.0 wt %.

### EXAMPLES

The present invention is explained in detail by referring to Examples, which are not to be construed as limitative.

[Quantitative Analysis Method]

The quantitative analysis of glutamic acid and pyrrolidonecarboxylic acid was performed by HPLC (column: “SUMIPAX PGODS 100-7” manufactured by Sumitomo Chemical Analysis Center, column temperature: 40° C., eluent: 10 mM aqueous phosphoric acid solution, flow rate: 1 ml/min, detector: UV, 210 nm). The quantity was determined by comparing comparative area with standard solutions containing glutamic acid (manufactured by Ajinomoto Co., Ltd.) and pyrrolidonecarboxylic acid (manufactured by ALDRICH), each solution being 100mg/dl, as standard.

[Optical Purity Analysis Method]

The optical purity of glutamic salt and zinc pyrrolidonecarbonate dihydrate was determine by HPLC (chiral column: “OA-5500” manufactured by Sumitomo Chemical Analysis center, Column temperature: room temperature, Eluent: aqueous copper(II) sulfate solution (2 mmol)/acetonitrile=95/5, flow rate: 1 ml/min, detection: UV 254 nm). An L/D is the ratio of area detected by the above analysis. The optical purity is calculated by subtracting % of racemate from the whole purity (100%). That is, the optical purity is a value calculated by 100%−2×percentage of D form.

[pH Measurement Method]

A pH was measured by using the Castani LAB pH meter(HORIBA Company, F-series, pH electrode: #6366-10 D, internal standard solution: saturated potassium chloride aqueous solution). Before using the pH meter, two-point calibration was performed with the standard solution having pH 6.86 (neutral phosphate solution) and the standard solution having pH 4.01 (phthalic acid salt) at 25° C. The preferred determining temperature of pH-correction was 25° C. However, a value of pH is not affected by temperature of system when change of pH value hardly occurs, namely, in the range of 25° C.±5° C. In the case of out of range of 25° C.±5° C. by condition of crystallization etc., measurement of pH must be performed by correcting conversion value by temperature factor, or by using the auto conversion function of the pH meter.
[Conversion formula of pH] pH(25)=pH(t)−α(t−25)
wherein, pH(25) is the pH conversed at 25° C.,
pH(t) is the pH at t° C.,
α is the temperature coefficient (pH/° C.), and
t is the temperature (° C.) on measuring.
   [Equation of temperature coefficient] α=(pH[25]−pH[t])/(t−25)
   wherein, pH[25]is the pH conversed at 25° C., and
pH[t] is the pH at t° C.
   [Solution Properties Analysis Method]

A measuring apparatus, condition, preparing method of samples and determining step is the followings;

1. Apparatus: Spectrophotometer V-570 (JAS. CO)

Spectrophotometer (UV/VIS/NIR)

2. Measurement Condition

light metering mode: % T

measure wavelength: 550 nm

band width: 2.0 nm

band width (near infrared): 8.0 nm

response: Medium

repeating times: 2 times

repeating interval: 3 sec.

measure cell: quartz glass cell T-1-UV-10 manufactured by TOSOH Quartz Co.

(Optical path width: 10 mm, outside width; 12.5 mm, height; 45 mm)

measure temperature: 25° C.(R.T.)

3. Sample Preparation Method

A 4 g of zinc pyrrolidonecarboxylate dihydrate was dissolved in water at R.T. to obtain the solution having constant volume of 40 ml (10 g/dl solution), and then placed on a thermostat of 25° C. for one day. This solution was used.

4. Measuring Step

1) Stabilization of value was performed, thereby starting setting until 2 hours before measuring.

2) Determination of measuring program: Fixed Wavelength program was selected, and then initialization of wavelength was performed.

3) Injecting ion exchange water (about 3 ml) into each of blank cell and sample cell, setting on photometer, correcting zero point, and then determining a blank (Completion of measuring preparation).

4) The solution prepared on the previous day was stirred for 1 min (Magnetic Stirrer, MITAMURA RIKEN KOGYO INC., Magmix Stirrer) at 3 of speed range, and then the solution (3 ml) was injected into measuring cell immediately.

5) The cell was equipped photometer immediately after confirming absence of bubble in the cell.

6) After setting, the cell was stood for 1 min, and then subjected to measurement.

7) The transmittance (% T) is the value measured with photometer.

Under the above conditions, the transmittance was obtained accurately although measuring sample comprised insoluble material, because insoluble material was dispersed uniformly in the whole cell. On the other hand, when the cell is not stirred or stood for not less than 10 minutes before measurement after setting, precipitation occurs to increase apparent transmittance, thus preventing correct value measurement.

5. Evaluation Criteria

In consideration of the relationship between visual dissolution state and transmittance, the evaluation criteria were as follows;
⊙: 100-98%
[symbol] : 97-94%
Δ: 93-85%
x: 84-0%

### EXAMPLE 1

Production of Zinc Pyrrolidonecarboxylate Dihydrate from Sodium L-glutamate

(pH is 3.7 Before Separating Crystal by Nitric Acid Addition)

In an autoclave, aqueous solution of sodium L-glutamate monohydrate (61.1 g) was heated at 180° C. for two hours to obtain 50 wt % aqueous solution of sodium pyrrolidonecarboxylate. To 100.0 g (0.33 mol, pH 7.7, optical purity 84%, L/D ratio=92/8) of the 50 wt % aqueous solution of sodium pyrrolidonecarboxylate, 2.7 g of nitric acid (purity 60 wt %) was added to adjust to pH 5.2. The aqueous solution in which 47.6 g (0.17 mol) of zinc sulfate 7-hydrate was dissolved in 34.2 g of water was added to the aqueous sodium pyrrolidonecarboxylate solution (pH 4.1). This was stirred for 30 min at room temperature (pH 3.7) to obtain crystal, and then filtered. The obtained crystal was washed with water (21.9 g) to obtain 32.0 g (0.09 mol, yield 55%) of zinc pyrrolidonecarboxylate dihydrate. Optical purity was 99.8% (L/D ratio 99.9/0.1).

### EXAMPLE 2

Producing Zinc Pyrrolidonecarboxylate Dihydrate from Sodium L-glutamate

(pH is 2.8 Before Separating Crystal by Nitric Acid Addition)

In an autoclave, aqueous solution of sodium L-glutamate monohydrate 61.1 g was heated at 180° C. for two hours to obtain 50 wt % aqueous solution of sodium pyrrolidonecarboxylate. To 100.0 g (0.33 mol, pH 7.7, optical purity 84%, L/D ratio=92/8) of the 50 wt % aqueous solution of sodium pyrrolidonecarboxylate, 11.5 g of nitric acid (purity 60 wt %) was added to adjust to pH 4.0. The aqueous solution in which 47.6 g (0.17 mol) of zinc sulfate 7-hydrate is dissolved in 34.2 g of water was added to the aqueous sodium pyrrolidonecarboxylate solution (pH 3.1). This was stirred for 30 min at room temperature (pH 2.8) to obtain crystal, and then filtered. The obtained crystal was washed with water (21.9 g) to obtain 25.1 g (0.07 mol, yield 43%) of zinc pyrrolidonecarboxylate dihydrate. Optical purity was 98.2% (L/D ratio 99.1/0.9).

### EXAMPLE 3

Producing Zinc Pyrrolidonecarboxylate Dihydrate from Sodium L-glutamate

(pH is 3.8 Before Separating Crystal from Nitric Acid Addition, PCANa: Optical Purity 72%, L/D=86/14)

To 78.0 g (0.25 mol. pH 7.7, optical purity 72%, L/D ratio=86/14) of the 50 wt % aqueous solution of sodium pyrrolidonecarboxylate, 2.7 g of nitric acid (purity 60 wt %) was added to adjust to pH 5.2. The aqueous solution in which 47.6 g (0.17 mol) of zinc sulfate 7-hydrate is dissolved in 34.2 g of water was added to the aqueous sodium pyrrolidonecarboxylate solution (pH 4.1). This was stirred for 30 min at room temperature (pH 3.8) to obtain crystal, and then filtered. The obtained crystal was washed with water (21.9 g) to obtain 32.0 g (0.09 mol, yield 50%) of zinc pyrrolidonecarboxylate dihydrate. Optical purity was 99.8% (L/D ratio 99.9/0.1).

### EXAMPLE 4

Producing Zinc Pyrrolidonecarboxylate Dihydrate from Sodium L-glutamate

(pH is 4.0 Before Separating Crystal by Nitric Acid Addition)

In an autoclave, aqueous solution of sodium L-glutamate monohydrate 61.1 g was heated at 180° C. for two hours to obtain 50 wt % aqueous solution of sodium pyrrolidonecarboxylate. To 100.0 g (0.33 mol, pH 7.7, optical purity 84%, L/D ratio=92/8) of the 50 wt % aqueous solution of sodium pyrrolidonecarboxylate, 1.5 g of nitric acid (purity 60 wt %) was added to adjust to pH 5.4. The aqueous solution in which 47.6 g (0.17 mol) of zinc sulfate 7-hydrate is dissolved in 34.2 g of water was added to the aqueous sodium pyrrolidonecarboxylate solution (pH 4.4). This was stirred for 30 min at room temperature (pH 4.0) to obtain crystal, and then filtered. The obtained crystal was washed with water (21.9 g) to obtain 33.8 g (0.09 mol, yield 58%) of zinc pyrrolidonecarboxylate dihydrate. Optical purity was 99.8% (L/D ratio 99.9/0.1).

### EXAMPLE 5

Producing Zinc Pyrrolidonecarboxylate Dihydrate from Sodium L-glutamate

(pH is 3.7 Before Separating Crystal by Zinc Sulfate Water and Nitric Acid Addition)

In an autoclave, aqueous solution of sodium L-glutamate monohydrate 61.1 g was heated at 180° C. for two hours to obtain 50 wt % aqueous solution of sodium pyrrolidonecarboxylate. The aqueous solution (pH 0.4) in which 2.7 g of nitric acid (purity 60 wt %) and 47.6 g (0.17 mol) of zinc sulfate 7-hydrate are dissolved in 34.2 g of water was added to 100.0 g (0.33 mol, pH 7.7, optical purity 84%, L/D ratio=92/8) of the 50 wt % aqueous solution of sodium pyrrolidonecarboxylate (pH 4.0). This was stirred for 30 min at room temperature (pH 3.7) to obtain crystal, and then filtered. The obtained crystal was washed with water (21.9 g) to obtain 32.1 g (0.09 mol, yield 55%) of zinc pyrrolidonecarboxylate dihydrate. Optical purity was 99.8% (L/D ratio 99.9/0.1).

### EXAMPLE 6

Producing Zinc Pyrrolidonecarboxylate Dihydrate from Sodium L-glutamate Salt

(pH is 6.0 Before Zinc Salt Addition and 4.0 Before Separating Crystal by Nitric Acid Addition)

In an autoclave, aqueous solution of sodium L-glutamate monohydrate 61.1 g was heated at 180° C. for two hours to obtain 50 wt % aqueous solution of sodium pyrrolidonecarboxylate. To 100.0 g (0.33 mol, pH 7.7, optical purity 84%, L/D ratio=92/8) of the 50 wt % aqueous solution of sodium pyrrolidonecarboxylate, 0.5 g of nitric acid (purity 60 wt %) was added to adjust to pH 6.0. The aqueous solution in which 47.6 g (0.17 mol) of zinc sulfate 7-hydrate is dissolved in 34.2 g of water was added to the aqueous sodium pyrrolidonecarboxylate solution (pH 4.9). This was stirred for 5 min at room temperature (pH 4.6), and then 0.9 g of nitric acid (purity 60 wt %) was added to adjust to pH 4.0. This was again stirred for 20 min at room temperature (pH 4.0) to obtain crystal, and then filtered. The obtained crystal was washed with water (21.9 g) to obtain 31.2 g (0.09 mol, yield 53%) of zinc pyrrolidonecarboxylate dihydrate. Optical purity was 99.8% (L/D ratio 99.9/0.1).

### EXAMPLE 7

Producing Zinc Pyrrolidonecarboxylate Dihydrate from Sodium L-glutamate

(pH is 3.7 Before Separating Crystal by Hydrochloric Acid Addition, and Zinc Chloride is Used as Zinc Source)

In an autoclave, aqueous solution of sodium L-glutamate monohydrate 48.0 g was heated at 180° C. for two hours to obtain 50 wt % aqueous solution of sodium pyrrolidonecarboxylate. To 78.0 g (0.26 mol, pH 7.7, optical purity 84%, L/D ratio=92/8) of the 50 wt % aqueous solution of sodium pyrrolidonecarboxylate, 1.9 g of hydrochloric acid (purity 36 wt %) was added to adjust to pH 5.0. The aqueous solution in which 18.1 g (0.13 mol) of zinc chloride is dissolved in 45.6 g of water was added to the aqueous sodium pyrrolidonecarboxylate solution (pH 4.1). This was stirred for 30 min at room temperature (pH 4.1) to obtain crystal, and then filtered. The obtained crystal was washed with water (17.3 g) to obtain 14.1 g (0.04 mol, yield 31%) of zinc pyrrolidonecarboxylate dihydrate. Optical purity was 99.8% (L/D ratio 99.9/0.1).

### COMPARATIVE EXAMPLE 1

Producing Zinc Pyrrolidonecarboxylate Dihydrate from Sodium L-glutamate

(pH is 5.4 Before Crystal Separation Without Nitric Acid Addition)

In an autoclave, aqueous solution of sodium L-glutamate monohydrate 61.1 g was heated at 180° C. for two hours to obtain 50 wt % aqueous solution of sodium pyrrolidonecarboxylate. The aqueous solution in which 47.6 g (0.17 mol) of zinc sulfate 7-hydrate is dissolved in 34.2 g of water was added to 100.0 g (0.33 mol, pH 7.7, optical purity 84%, L/D ratio=92/8) of the 50 wt % aqueous solution of sodium pyrrolidonecarboxylate (pH 5.3). This was stirred for 30 min at room temperature (pH 5.4) to obtain crystal, and then filtered. The obtained crystal was washed with water (21.9 g) to obtain 35.0 g (0.10 mol, yield 60%) of zinc pyrrolidonecarboxylate dihydrate. Optical purity was 99.8% (L/D ratio 99.9/0.1).

### COMPARATIVE EXAMPLE 2

Producing Zinc Pyrrolidonecarboxylate Dihydrate from Sodium L-glutamate

(pH is 4.6 Before Separating Crystal by Nitric Acid Addition)

In an autoclave, aqueous solution of sodium L-glutamate monohydrate 61.1 g was heated at 180° C. for two hours to obtain 50wt % aqueous solution of sodium pyrrolidonecarboxylate. To 100.0 g (0.33 mol, pH 7.7, optical purity 84%, L/D ratio=92/8) of the 50 wt % aqueous solution of sodium pyrrolidonecarboxylate, 0.5 g of nitric acid (purity 60 wt %) was added to adjust to pH 6.0. The aqueous solution in which 47.6 g (0.17 mol) of zinc sulfate 7-hydrate is dissolved in 34.2 g of water was added to 50 wt % aqueous solution of the sodium pyrrolidonecarboxylate (pH 4.8). This was stirred for 30 min at room temperature (pH 4.6) to obtain crystal, and then filtered. The obtained crystal was washed with water (21.9 g) to obtain 34.8 g (0.10 mol, yield 59%) of zinc pyrrolidonecarboxylate dihydrate. Optical purity was 99.8% (L/D ratio 99.9/0.1).

### COMPARATIVE EXAMPLE 3

Producing Zinc Pyrrolidonecarboxylate Dihydrate from Sodium L-glutamate

(pH is 4.3 Before Separating Crystal by Nitric Acid Addition)

In an autoclave, aqueous solution of sodium L-glutamate monohydrate 61.1 g was heated at 180° C. for two hours to obtain 50 wt % aqueous solution of sodium pyrrolidonecarboxylate. To 100.0 g (0.33 mol, pH 7.7, optical purity 84%, L/D ratio=92/8) of the 50 wt % aqueous solution of sodium pyrrolidonecarboxylate, 0.8 g of nitric acid (purity 60 wt %) was added to adjust to pH 5.6. The aqueous solution in which 47.6 g (0.17 mol) of zinc sulfate 7-hydrate is dissolved in 34.2 g of water was,added to the aqueous sodium pyrrolidonecarboxylate solution (pH4.6). This was stirred for 30 min at room temperature (pH 4.3) to obtain crystal, and then filtered. The obtained crystal was washed with water (21.9 g) to obtain 34.5 g (0.10 mol, yield 59%) of zinc pyrrolidonecarboxylate dihydrate. Optical purity was 99.8% (L/D ratio 99.9/0.1).

### COMPARATIVE EXAMPLE 4

Producing Zinc Pyrrolidonecarboxylate Dihydrate from Sodium L-glutamate

(pH of Solution is 5.2 by Nitric Acid Addition, and pH is 5.8 Before Separating Crystal by NaOH Addition)

In an autoclave, aqueous solution of sodium L-glutamate monohydrate 61.1 g was heated at 180° C. for two hours to obtain 50 wt % aqueous solution of sodium pyrrolidonecarboxylate. To 100.0 g (0.33 mol, pH 7.7, optical purity 84%, L/D ratio=92/8) of the 50 wt % aqueous solution of sodium pyrrolidonecarboxylate, 2.4 g of nitric acid (purity 60 wt %) was added to adjust to pH 5.2. The aqueous solution in which 47.6 g (0.17 mol) of zinc sulfate 7-hydrate was dissolved in 34.2 g of water was added to the aqueous sodium pyrrolidonecarboxylate solution (pH 4.1). Before precipitating crystal, 3.5 g of 27% aqueous sodium hydroxide solution was added thereto to adjust to pH 5.2. This was stirred for 30 min at room temperature (pH 5.8) to obtain crystal, and then filtered. The obtained crystal was washed with water (21.9 g) to obtain 34.9 g (0.10 mol, yield 60%) of zinc pyrrolidonecarboxylate dihydrate. Optical purity was 99.8% (L/D ratio 99.9/0.1).

TABLE 1

| analysis object | analysis item | Ex. 1 | Ex. 2 | Ex. 3 | Ex. 4 | Ex. 5 | Ex. 6 | Ex. 7 | Com. Ex. 1 | Com. Ex. 2 | Com. Ex. 3 | Com. Ex. 4 |
|---|---|---|---|---|---|---|---|---|---|---|---|---|
| (starting material) | optical purity (%) | 84.0 | 84.0 | 72.0 | 84.0 | 84.0 | 84.0 | 84.0 | 84.0 | 84.0 | 84.0 | 84.0 |
| sodium | | | | | | | | | | | | |
| pyrrolidone- | | | | | | | | | | | | |
| carboxylate (aq) | | | | | | | | | | | | |
| second step | pH before zinc salt | 5.2 | 4.0 | 5.2 | 5.4 | 7.7 | 6.0 | 5.0 | 7.7 | 6.0 | 5.6 | 5.2 |
| | addition | | | | | | | | | | | |
| | temperature of zinc | room | room | room | room | room | room | room | room | room | room | room |
| | salt exchange | temp. | temp. | temp. | temp. | temp. | temp. | temp. | temp. | temp. | temp. | temp. |
| | reaction | | | | | | | | | | | |
| | pH after zinc salt | 3.7 | 2.8 | 3.8 | 4.0 | 3.7 | 4.9 | 4.1 | 5.4 | 4.6 | 4.3 | 5.8 |
| | addition | | | | | | | | | | | |
| | pH before crystal | 3.7 | 2.8 | 3.8 | 4.0 | 3.7 | 4.0 | 4.1 | 5.4 | 4.6 | 4.3 | 5.8 |
| | separation | | | | | | | | | | | |
| zinc pyrrolidone- | optical purity (%) | 99.8 | 98.2 | 99.8 | 99.8 | 99.8 | 99.8 | 99.8 | 99.8 | 99.8 | 99.8 | 99.8 |
| carboxylate | yield (%) | 55 | 43 | 50 | 58 | 55 | 53 | 31 | 60 | 59 | 59 | 60 |
| dihydrate (product) | transmittance (T %) | 96.6 | 97.6 | 96.9 | 97.6 | 97.9 | 86.1 | 95.7 | 17.5 | 30.6 | 60.3 | 19.9 |
| | of 10 g/dl aqueous | | | | | | | | | | | |
| | solution | | | | | | | | | | | |
| | liquid of 10 g/dl | ◯ | ⊚ | ◯ | ⊚ | ⊚ | Δ | ◯ | X | X | X | X |
| | aqueous solution | | | | | | | | | | | |

*In all Examples, the starting material of zinc is zinc sulfate 7-hydrate
Ex. 1: pH before crystal separation by addition of nitric acid: 3.7
Ex. 2: pH before crystal separation by addition of nitric acid: 2.8
Ex. 3: pH before crystal separation by addition of nitric acid: 3.8, PCANa of starting material = L86/D14
Ex. 4: pH before crystal separation by addition of nitric acid: 4.0
Ex. 5: pH before crystal separation by addition of zinc sulfate and nitric acid mixture: 3.7
Comp. Ex. 1: without addition of nitric acid, pH before crystal separation: 5.4
Comp. Ex. 2: pH before crystal separation by addition of nitric acid: 4.6
Comp. Ex. 3: pH before crystal separation by addition of nitric acid: 4.3
Comp. Ex. 4: pH 5.2 by nitric acid addition, pH before crystal separation by NaOH: 5.8

As illustrated in the Table, water-soluble zinc salt was added to the aqueous sodium pyrrolidonecarboxylate solution and pH was controlled to fall within the range of 2.8 to 4.2, and then the precipitated crystal was collected, thereby conveniently producing zinc pyrrolidonecarboxylate dihydrate with higher purity.

### EXAMPLE 8

Moisture Gel Preparation Example

Using zinc pyrrolidonecarboxylate dihydrate obtained in Example 1, moisture gel is manufactured as a cosmetic through the following procedure:

| Dipropyleneglycol | 7.0 wt % |
|---|---|
| PEG1500 | 8.0 |
| Carboxyvinyl polymer | 0.4 |
| Methyl cellulose | 0.2 |
| POE(15)oleyl alcohol ether | 1.0 |
| Potassium hydroxide | 0.1 |
| zinc pyrrolidonecarboxylate | 0.1 |
| dihydrate (Example 1) | |
| purified water | 83.2 |

Such moisture gel is colorless and transparent and presents sensation of coolness and refreshing.

Among various salts, zinc pyrrolidonecarboxylate is very useful compounds industrially presenting broader functions such as the astringent effect, bacteriostatic action, an odor prophylaxis component, a production intermediate for an optical active glutamic acid, and a stabilizing effect of polyvinyl chloride resin.

This application is based on a patent application No. 129063/2004 filed in Japan, the contents of which are hereby incorporated by reference.

1. A method of producing zinc pyrrolidonecarboxylate dihydrate, which comprises the steps of
adding a zinc salt to a solution of a salt of pyrrolidonecarboxylic acid in an aqueous medium to allow reaction of the zinc salt with the salt of pyrrolidonecarboxylic acid to give precipitated crystals of zinc pyrrolidonecarboxylate dihydrate in the aqueous medium, and separating the crystals of zinc pyrrolidonecarboxylate dihydrate from the aqueous medium at a pH ranging from 2.8 to 4.2 (conversion value at 25° C.).

2. The production method of claim 1, wherein the salt of pyrrolidonecarboxylic acid has an optical purity ranging from 100% to 50%.

3. The production method of claim 2, fturther comprising a step of obtaining the salt of pyrrolidonecarboxylic acid with an optical purity ranging from 100% to 50%, by heating a salt of glutamic acid with an optical purity ranging from 100 to 50%, in the aqueous medium.

4. The production method of claim 1, wherein the zinc salt is at least one selected from the group consisting of a zinc chloride and a zinc sulfate.

5. A zinc pyrrolidonecarboxylate dihydrate with an optical purity ranging from 100 to 95%, which is obtained by the production method of claim 1.

6. A method of evaluating a zinc pyrrolidonecarboxylate dihydrate, which comprises a step of measuring light transmittance of a 10 g/dl aqueous solution thereof.

7. The zinc pyrrolidonecarboxylate dihydrate of claim 5, wherein the light transmittance of a 10 g/dl aqueous solution thereof at a wavelength of 550 nm is within the range of 94 to 100% under an optical path length of 10 mm.

8. A zinc pyrrolidonecarboxylate dihydrate with an optical purity ranging from 100% to 95%, wherein a light transmittance of a 10 g/dl aqueous solution thereof at a wavelength of 550 nm is within the range of 94 to 100% under an optical path length of 10 mm.

9. A cosmetic comprising the zinc pyrrolidonecarboxylate dihydrate of claim 5.

10. A cosmetic comprising the zinc pyrrolidonecarboxylate dihydrate of claim 8.

11. The production method of claim 2, wherein the zinc salt is at least one selected from the group consisting of a zinc chloride and a zinc sulfate.

12. The production method of claim 3, wherein the zinc salt is at least one selected from the group consisting of a zinc chloride and a zinc sulfate.

13. A zinc pyrrolidonecarboxylate dihydrate with an optical purity ranging from 100 to 95%, which is obtained by the production method of claim 2.

14. A zinc pyrrolidonecarboxylate dihydrate with an optical purity ranging from 100 to 95%, which is obtained by the production method of claim 3.

15. A zinc pyrrolidonecarboxylate dihydrate with an optical purity ranging from 100 to 95%, which is obtained by the production method of claim 4.
